(19) United States
(12) Patent Application Publication
(10) Pub. No.: US 2008/0227757 A1
(43) Pub. Date: Sep. 18, 2008

(54) Formulation for dermal application

(76) Inventors: Christel Muller-Goymann, Braunschweig (DE); Nadja Gruning, Braunschweig (DE)

Correspondence Address: COLLARD & ROE, P.C., 1077 NORTHERN BOULEVARD, ROSLYN, NY, 11576, US

(21) Appl. No.: 11/411,476

(22) Filed: Apr. 26, 2006

(30) Foreign Application Priority Data
Apr. 26, 2005 (DE) 10 2005 019 628.4

(51) Int. Cl. A61K 31/197; A61K 31/4409; A61K 31/4164; A61K 31/616; A61K 31/57

(52) U.S. Cl. 514/162; 514/561; 514/329; 514/396; 514/171

(57) ABSTRACT

The present invention relates to formulations on an aqueous basis with polyetherpolyol, dimethyl isosorbide, lipid and alcohol content suitable for penetration reinforcement of pharmaceutical active ingredients in the dermal application. The pharmaceutical active ingredients can be amphiphilic, zwitterionic, strongly polar or lipophilic. A preferred active ingredient is 5-aminolevulinic acid.

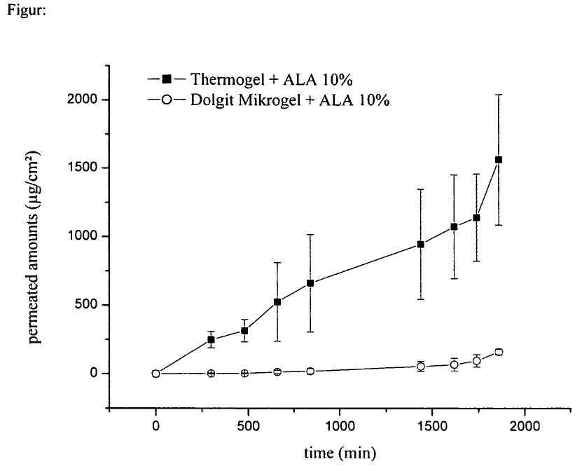

The present invention relates to formulations, which are suitable for penetration reinforcement of amphiphilic, zwitterionic, polar or lipophilic pharmaceutical active ingredients in dermal and/or transdermal application.

Dermal application relates in particular to a formulation for the active ingredient 5-aminolevulinic acid as a key substance in Photodynamic Therapy (PDT), in which the formation of highly reactive oxygen species, catalysed by porphyrin systems, for example singlet oxygen, peroxide radicals, hydroxyl radicals and superoxide radical anions as well as hydrogen peroxide is utilised by means of energy irradiated as light.

Medical indications for the inventive formulation of 5-aminolevulinic acid (5-ALA) are thus in particular actinic keratoses as a constituent of Photodynamic Therapy, in which light in the region of ca. 630 nm is irradiated locally or laser-focussed following application, and acne.

### PRIOR ART

Photodynamic tumour therapy utilises photosensitisers, which preferably have high tumour selectivity, minimal cytotoxicity, brief dwell time in tissue, high yield of singlet oxygen and adequate photostability. These are in particular photosensitisers, which are concentrated primarily in tumour tissue, so that the latter is damaged directly with local radiation. Compounds containing porphyrin or their precursors in biosynthesis, such as for example 5-aminolevulinic acid or its derivatives, are used as photosensitisers.

Oligomer hematoporphyrin ether is used to treat non-small-cell bronchial carcinoma of the oesophagus carcinoma and bladder carcinomas. Activating occurs via laser radiation on a wavelength of 630 nm. Hematoporphyrin is used for treating alopecia for example, resulting from carefully eliminating iron from heme.

As an alternative to porphyrin systems 5-aminolevulinic acid is used, which is converted biosynthetically in the cell into protoporphyrin IX, which then becomes heme by intercalation of Fe²⁺. A pharmaceutical preparation, Metvix®, uses the methyl ester of 5-aminolevulinic acid due to its character, more lipophilic compared to 5-aminolevulinic acid, increasing permeation through the layers of skin. This derivative of 5-aminolevulinic acid is applied to photodynamic therapy of actinic keratoses on the face and head, as well as of superficial and nodular basal cell carcinomas. The applied 5-aminolevulinic acid, or respectively its methyl ester, is concentrated in the epithelial cells and is converted to protoporphyrin IX, a strong photosensitiser. Radiation is conducted on a wavelength of 570 to 670 nm. (German Pharmacist Bulletin, No. 27, pages 3362-3368).

EP 0 215 423 B1 describes transdermal resorbable formulations of aryl propionic acids for its systemic administration, which contain with 1-15% by weight active ingredient, 10-40% by weight poloxamer, 10-50% by weight organic solvent and at least 10% by weight water. Examples of organic solutions are ethanol, isopropanol, 1,2-propane diol, glycerin, isosorbide, dimethyl isosorbide, polyoxyethylene(4)-lauryl ether, polyoxypropylene-(15)-stearyl ether and DMSO. The examples mostly contain ibuprofen, in two cases alternatively ketoprofen or naproxen. No lipid content is mentioned.

Squillante et al. (European Journal of Pharmaceutics and Biopharmaceutics, 265-271 (1998)) describe the codiffusion of propylene glycol and dimethyl isosorbide (DMIS) through mouse skin in the presence of oleic acid, and permeation of nifedipine from a mixture also containing propylene glycol and dimethyl isosorbide, in addition to oleic acid, known as a penetration improver. This establishes that oleic acid promotes permeation of dimethyl isosorbide, however DMIS has no permeation-promoting effect for propylene glycol and oleic acid. On this finding of the codiffusion of propylene glycol and DMIS the authors postulate a carrier mechanism for the drug nifedipine, since it has good solubility both in propylene glycol and in DMIS. Beyond the known solvent effect of propylene glycol and DMIS there is no permeation-promoting effect shown in the publication.

Lee et al. (International Journal of Pharmaceutics, 105-114 (1997)) describe a formulation of a synthetic capsaicin analog, which has 20 to 30% by weight poloxamer, 20 to 40% by weight ethanol, 0 to 20% by weight, propylene glycol and 5% by weight isopropyl myristate as fatty acid in water.

The Red List 2005 (Pharmaceutical Index for Germany, Editio Cantor Verlag Aulendorf) publishes a compound under the name dolgit microgel with 5% by weight ibuprofen in 2-propanol, dimethyl isosorbide, poloxamer, medium-chain triglycerides, lavender oil and neroli oil in water.

Winkler and Müller-Goymann (European Journal of Pharmaceutics and Biopharmaceutics, 427-437 (2005)) compare the permeation reinforcement of dolgit microgel, excipial cream or basic cream on 5-aminolevulinic acid and 5-aminolevulinic acid butyl ester. With these formulations only dolgit microgel exhibited a poloxamer content. When compared to permeation of 5-aminolevulinic acid and its butyl ester through isolated human stratum corneum epidermidis it became evident that permeation through human skin was most effectively reinforced by a formulation of butyl ester in dolgit microgel (containing ibuprofen). Trials conducted with modified formulations to attain an improvement in permeation of the ALA-butyl ester by waiving the active ingredient ibuprofen and/or medium-chain triglyceride, as well as by variation of the proportions of the other constituents, fell through. In this context Winkler and Müller-Goymann conclude that the fluidising and permeation-reinforcing effect of dolgit microgel is essentially traced back to the ibuprofen acid content, while a lesser permeation-reinforcing effect is ascribed to medium-chain triglycerides.

Further interim trials by the inventor have additionally shown that the permeation-reinforcing effect of medium-chain triglycerides for 5-aminolevulinic acid-butyl ester in dolgit microgel occurs as preferred formulation essentially only in combination with ibuprofen content.

### OBJECT OF THE INVENTION

Before the known state of the art the object of the present invention is to provide a compound, with which the application or respectively the dermal transport of 5-ALA can be modified or respectively strengthened, resulting-in increased concentrations of 5-ALA in the target cells.

A further task of the present invention is to provide a compound, with which the transport, i.e. the permeation, of amphiphilic, zwitterionic or strongly polar active ingredients through the human Stratum corneum can be reinforced, particularly preferably with salve-like consistency at room and/or body temperature.

For medical application it is significant that the formulation for 5-aminolevulinic acid at body temperature has sufficiently high viscosity, such that the active ingredient remains at the application site where it can permeate. At these application temperatures highly fluid formulations are accordingly unsuitable. Due to the instability of 5-aminolevulinic acid (5-ALA), as compared to oxygen and thus also compared to air, it is further preferred to provide an ointment base, which can be produced separately, i.e. without the active ingredient, and stored, and to which the active ingredient, e.g. 5-aminolevulinic acid, can also be added homogeneously.

At the same time the formulation can still promote the permeation of 5-aminolevulinic acid through the epithelium, for example human skin, and, particularly preferably contain no active ingredients other than 5-aminolevulinic acid.

According to pretrial findings, disclosing that in known formulations consistency is influenced essentially by ibuprofen and this active ingredient should not be contained regularly as formulation aid, the object of the present invention in particular is to provide an ibuprofen-free formulation for improving the permeation of amphiphilic, zwitterionic, strongly polar or lipophilic pharmaceutical active ingredients, in particular 5-aminolevulinic acid.

A further task of the present invention is to provide a pharmaceutical compound for treating actinic keratoses and epithelial cell carcinomas, as well as acne.

### GENERAL DESCRIPTION OF THE INVENTION

The present invention solves the above tasks by providing a formulation, with which pharmaceutical active ingredients, in particular 5-ALA, can be infiltrated through human skin in a large quantity. In the preferred embodiment the inventive formulation is thermoreversibly gelling, i.e. for the purposes of the invention, at body to room temperature the formulation is spreadable to solid, and at temperatures of ca. 12° C. it has a lower viscosity, i.e. is more liquid. The inventive formulation is stable under storage conditions at temperatures below body temperature, for example from ca. 5 to 12° C., and has a low viscosity so that an active ingredient such as 5-aminolevulinic acid can be distributed therein homogeneously by means of a simple stirrer, for example an Unguator®. The homogeneous distribution of an active ingredient at room temperature in the formulation is likewise possible by means of high-speed stirrer units, for example an Unguator®. At application temperature, i.e. at approximately body temperature, the formulation has an active ingredient independent of content, e.g. with or without 5-ALA, clearly higher viscosity, namely a salve-like consistency. This higher viscosity suffices to keep the active ingredient on the skin for a sufficiently long time (for example from 1 to 5 h) for permeation, so that the permeation-reinforcing effect of the active ingredient occurs at the application site. The inventive formulation has a substantially colloidal structure.

The inventive formulation preferably has no ibuprofen content, since this active ingredient is not required for adjusting the viscosity or permeation properties.

The present invention also provides a manufacturing process, enabling the inventive formulation to be manufactured easily, before the active ingredient is finally added.

In a preferred embodiment the invention provides a formulation of 5-ALA or another active ingredient, resulting in an increase in concentration of the active ingredient in the target tissue following topical application of the formulation.

The inventive formulation has an aqueous base, containing

0.1-20% by weight at least of an amphiphilic, zwitterionic or strongly polar pharmaceutical active ingredient, preferably 5-aminolevulinic acid,

10-30% by weight of a polyetherpolyol or a mixture thereof, for example selected from polyethylene glycol (α-hydro-ω-hydroxy-poly(oxy-1,2-ethane diol)), polypropylene glycol and/or poloxamer (particularly preferably poloxamer 407, α-hydro-ω-hydroxy-poly(oxyethylene)-poly(oxypropylene)-poly(polyoxyethylene)-block copolymer,

5-25% by weight dimethyl isosorbide,

2.5-10% by weight triglyceride, preferably medium-chain triglyceride, in particular according to the German Pharmacopoeia, and

5-20% by weight of a C₂- to C₄-alcohol or a mixture thereof, preferably selected from among ethanol and isopropanol,

remainder water,

preferably without ibuprofen.

### EXACT DESCRIPTION OF THE INVENTION

The present invention provides a formulation with penetration-reinforcing effect for amphiphilic, zwitterionic or strongly polar pharmaceutical active ingredients, suitable in particular for topical application of the pharmaceutical active ingredients to skin epithelium, which is solid at room temperature or respectively semisolid and is liquid at lower temperatures, for example at 5-12° C. For the purposes of the invention this behaviour of the temperature-dependent viscosity change is designated as thermoreversibly gelling.

The inventive formulation provides a matrix, which can integrate amphiphilic, zwitterionic and also highly polar active ingredients and can have the latter, preferably 5-aminolevulinic acid, permeate through the human skin with a high degree of efficacy.

The inventive formulation has polyetherpolyol in the region of 10-30% by weight, preferably 15-25% by weight, particularly preferably 20% by weight. The polyetherpolyol can be selected from among polyoxyethylene glycols having a molecular weight in the region of 1000 to 30000, polyoxypropylene glycols in the region of 1000 to 30000, polyoxyethylene-polyoxypropylene block copolymers with a molecular weight in the region of 1000 to 30000 and mixtures thereof. Polyethylene glycols can also be used as polyoxyethylene glycol, preferably for example polyethylene glycol 600 (PEG-12), polyethylene glycol 400 (PEG-8) and polyethylene glycol 300 (PEG-6).

Particularly preferred polyetherpolyols are poloxamers with a molecular weight in the region of ca. 2000 to 20000 with an oxyethylene group portion in the region of 40 to 90% by weight, for example poloxamer 124, poloxamer 188, poloxamer 237 and poloxamer 338, as specified in Ph. Eur. A particularly preferred polyetherpolyol is poloxamer 407.

The inventive formulation also contains dimethyl isosorbide in the region of 5-25% by weight, preferably in the region of 10-20% by weight or up to 15% by weight, particularly preferably 12-13% by weight.

A further constituent of the inventive formulation is lipids, selected from among medium-chain triglycerides, fatty oils, paraffin oil, liquid waxes, isopropyl myristate, cetyl oleate and mixtures thereof in the region of 2.5-10% by weight, preferably 4-6% by weight. Particularly preferable are medium-chain triglycerides, for example corresponding to the definition of the European Pharmacopoeia (Ph. Eur., 4th edition, Grundwerk 2002), i.e. a mixture of triglycerides of saturated fatty acids, mainly caprylic acid and caprinoic acid, having at least 95% saturated fatty acids with 8 to 10 carbon atoms. A fatty acid fraction of the following composition is particularly preferred: capronoic acid at a maximum of 2%, caprylic acid at 50 to 80%, caprinic acid at 20 to 50%, laurinic acid at the most at 3% and myristinic acid at the most at 1%.

The inventive formulation further contains ca. 5-20% by weight, preferably ca. 10-15% by weight, particularly preferably ca. 12-13% by weight of a C₂- to C₄-alcohol, preferably ethanol, propanol, butanol, tert. butanol, particularly preferably isopropanol.

The inventive compound also contains ca. 40-60% by weight water, preferably ca. 45-55% by weight water, particularly preferably ca. 50% by weight water.

Amphiphilic, zwitterionic and strongly polar active ingredients in particular can be integrated into the inventive formulation as active ingredients. A preferred active ingredient according to the present invention is 5-aminolevulinic acid in a proportion of ca. 0.1-20% by weight, particularly preferably ca. 5-15% by weight, most preferably ca. 10% by weight of the overall composition.

The inventive production process is based on mixing formulation constituents, by way of advantage without the necessity for any preprocessing steps of individual constituents, for example heating for liquefaction.

The polyetherpolyol can be present as a solid, to which dimethyl isosorbide (liquid) is then added, followed by a lipid, for example medium-chain triglyceride (liquid). Next, the C₂- to C₄-alcohol (liquid) is added, and finally water. The inventive formulation can be produced by simple mechanical mixing at room temperature, for example stirring with a mechanical high-speed agitator, e.g. an Unguator®, with the mixture gelling immediately during the stirring procedure.

The mixed, gelled preparation is stable under storage conditions e.g. overnight or longer at room temperature. Active ingredients for different indications can be worked in by stirring: for example 5-aminolevulinic acid (for PDT), bamipine maleate (antihistaminic), clotrimazole, terbinafine (both antimycotics), acetylsalicylic acid (non-steroidal antirheumatic and analgetic) or hydrocortisone acetate (antiphlogistic).

The inventive production process manages advantageously with a single high-speed agitator for producing the mixture, for example an Unguator suitable for pharmacies, so that the formulation can also be produced in quantities without additional requirement for equipment. The simple production process of the invention also offers advantages in industrial-scale production, since no constituents have to be particularly preprocessed, and heating for example can dispense with liquefaction.

By way of alternative the production process can comprise the steps of combining the formulation constituents polyetherpolyol, dimethyl isosorbide, C₂-C₄ alcohol and water, therefore still without lipid or active ingredient, in a container, and be carried out for example in a mortar. During storage at low temperature, e.g. 4° C. overnight, a clear viscous solution results. The lipid portion can be added to this solution, preferably after heating to room temperature, and incorporated by stirring. The mixture becomes cloudy and with further heating, e.g. by stirring, the mixture solidifies into a thermoreversible gel. The active ingredient can be incorporated into this gel by stirring.

The inventive formulation has the particular advantage of amphiphilic or zwitterionic and also strongly polar active ingredients being able to be incorporated directly, without derivatisation required, to obtain a formulation of the active ingredient suited to permeation via epithelium. For example, 5-aminolevulinic acid without derivatisation to its methyl ester can be incorporated into the inventive thermoreversibly gelling formulation and permeated through the skin after topical application in a quantity sufficiently large for therapy.

A further advantage of the present invention is that some active ingredients can be used in a more cost-effective chemical structure, which itself has not been adapted via special derivatisation for permeation through epithelium. For example, 5-aminolevulinic acid can be used as such as active ingredient, and can be obtained substantially more cost-effectively than its methyl ester permeating through the skin, namely approximately by a factor of 10.

Due to more effective permeation of the active ingredient in the skin the reaction time can be curtailed, or respectively the active ingredient concentrations permeated in the skin during the same reaction time are higher. A shorter reaction time of 5-ALA can be applied for photodynamic treatment, or more effective reduction of cells during radiation can be obtained due to higher permeation-rates.

The inventive formulation may be stored and thus can advantageously be produced independently of the active ingredient to be incorporated later, and stored without cooling, e.g. above 13° C. Due to thermoreversible gelling of the inventive formulation, introducing active ingredients is easy to manage, since the active ingredients can be integrated into the formulation by simple mechanical stirring. If preferred, the viscosity of the inventive formulation can be lowered by cooling, for example to temperatures of 5-12° C. Heating the formulation containing active ingredients to room temperature after an active ingredient is mixed in, produces the desired viscosity of creamy or semisolid to solid, without the need for further procedural steps. This rise in viscosity at higher temperatures is independent of the active ingredient mixed in.

In the exact description of the invention 5-aminolevulinic acid is used in the inventive formulation as an example for a pharmaceutically active ingredient. At the same time reference is made to the figure showing the permeation of 5-aminolevulinic acid in the inventive thermogel (▪) and for comparison in dolgit microgel (o).

### EXAMPLE 1

### Production of the Inventive Formulation with Poloxamer

The following constituents are weighed out in a conventional Unguator suitable for pharmacies or mortar as mixing vessel at room temperature:

20.0 g poloxamer 407 (Lutrol F 127, Bayer), 12.5 g dimethyl isosorbide (liquid), 5.0 g medium-chain triglyceride (liquid, corresponding to European Pharmacopoeia, Ph. Eur.), 12.5 g isopropanol and finally 50.0 g water.

A mechanical stirrer is inserted through the opening in the storage vessel and the combined ingredients are mixed by stirring. The at first liquid mixture gels immediately from stirring at ca. 1450 Upm for 1.5 min.

The ready-mixed, gel-like preparation is stored overnight at room temperature. The pH of the solution or respectively the gel-like mixture can be adjusted to any desired value.

### EXAMPLE 2

### Formulation with Poloxamer and 5-Aminolevulinic Acid as Active Ingredient

10% by weight 5-aminolevulinic acid is incorporated into an inventive formulation produced according to Example 1 by stirring with an Unguator®.

### EXAMPLE 3

### Measuring of the Permeation of Active Ingredient from the Inventive Formulation Through Human Stratum Corneum

To determine the permeation of an active ingredient from the inventive formulation compared to conventional formulations containing active ingredients pharmaceutical preparations containing 5-aminolevulinic acid were tested in vitro on isolated human skin.

Human Stratum corneum, originating from biopsies of plastic operations, was used as isolated human skin. Following the biopsy subcutaneous fat was removed and the skin was frozen in liquid nitrogen, then stored at −25° C. After gradual thawing the Stratum corneum was isolated by trypsination on filter paper, and impregnated with an aqueous 2% trypsin solution. After incubation at 37° C. for 24 h the Stratum corneum could be lifted off, incubated in a 0.01% aqueous solution trypsin inhibitor and then washed several times in water. This isolated horny layer was dried and stored at room temperature in the desiccator. For permeation examinations a modified Franz cell was used, the compartments of which were separated off through the Stratum corneum. Pretreatment of the Stratum corneum is done by total hydration in water and placement on a polycarbonate filter (Isopor membrane filter, type TMTP, 5.0 μm, Millipore) to boost mechanical stability. The donor compartment was filled with inventive or respectively comparative formulation containing 5-aminolevulinic acid, whereby 5-aminolevulinic acid at 10% by weight was contained in the formulations. The formulation was produced corresponding to Example 2 in the respective compound free of active ingredient.

The preparation containing 5-aminolevulinic acid, procurable according to Example 2, was tested for permeation as inventive preparation, as compared to a formulation with conventional cream base according to the German Pharmacopoeia (70 parts water, 30 parts hydrophilic salve from 30 parts emulsifying cetylstearyl alcohol (type A), 35 parts viscid paraffin and 35 parts soft Vaseline), and in comparison to a formulation in dolgit microgel (5% by weight ibuprofen, in 2-propanol, dimethyl isosorbide, poloxamer, medium-chain triglycerides, lavender oil, neroli oil and water).

After application the quantity of active ingredient permeated through the skin was measured. Compared to the conventional cream base the inventive formulation of 5-aminolevulinic acid brought an increase in the rate of permeation through the Stratum corneum by at least a factor of 10 to 20.

The permeation behaviour of 5-aminolevulinic acid in formulations containing poloxamer is shown in the attached figure, which clarifies that the inventive formulation (▪, thermogel+10% ALA) enables clearly stronger permeation through the human Stratum corneum over the measuring period than the dolgit microgel formulation (o), whereby this formulation contains the additional active ingredient ibuprofen, essential also for consistency at room or respectively body temperature. The material flow of 5-ALA was determined for the inventive formulation at 121×10⁻¹⁰±23.9×10⁻¹⁰ g/cm²s, the permeation coefficient at 937×10⁻¹⁰±1.85×10⁻⁸ cms. For the comparative composition in dolgit microgel the values for the material flow were 16.4×10⁻¹⁰±3.69×10⁻¹⁰ g/cm²s, or respectively the permeation coefficients were 147.0×10⁻¹⁰±33.0×10⁻¹⁰ g×cms.

The inventive thermogel results in a permeated material flow (flux) of the active ingredient, here represented by way of example by 5-ALA, which is greater by a factor of ca. 7.5 than according to the known state of the art, namely from a formulation such as dolgit microgel.

The improvement in permeation of the active ingredient from the inventive formulation could be ascertained also for other amphiphilic, zwitterionic, strongly polar or lipophilic active ingredients, in particular for bamipine maleate, clotrimazole, terbinafine, acetylsalicylic acid or hydrocortisone acetate. This observation is at the present attributable to the fact that for the abovementioned active ingredients the values for lipophily are in the proximity of those for 5-aminolevulinic acid and its esters. Corresponding to the log P values of measurements of the octanol-water distribution coefficients (P), employed to determine lipophily, high permeation through the human Stratum corneum could also be ascertained for these active ingredients in the inventive formulation.

The lipophily value P gives low lipophility at a low value of the logarithm of P (log P), and a high value of the log P gives high lipophily. The distribution coefficient P is measured at pH 7.4 and 21° C. in octanol water.

For 5-ALA the log P=−1.51692, for 5-ALA-methylester=−0.94233, for 5-ALA-ethylester=0.84113, for 5-ALA-butylester=1.42315, for 5-ALA-hexylester=1.83883, for 5-ALA-cyclohexylester=1.49392 and for 5-ALA-octylester=2.6199. Accordingly, a log P of −1.15 for the acetylsalicylic acid, of 2.18 for hydrocortisone acetate, of 3.30 for terbinafine and of 4.10 for clotrimazole is also an indication that active ingredients with log P values exhibit suitable lipophily in the water-octanol system at pH 7.4, T=21° C. in the region of −2 to 5, preferably from −1.6 to 4.5, to achieve high permeation through the human skin with the inventive formulation.

1. A pharmaceutical formulation, characterised in that it comprises
0.1-20% by weight 5-aminolevulinic acid,
10-30% by weight polyetherpolyol,
2.5-10% by weight lipids,
5-25% by weight dimethyl isosorbide,
5-20% by weight C₂-C₄-alcohol and
40-60% by weight water.

2. The pharmaceutical formulation as claimed in claim 1, characterised in that instead of or in addition to 5-aminolevulinic acid it contains another amphilphilic, zwitterionic, strongly polar or lipophilic pharmaceutical active ingredient.

3. The pharmaceutical formulation as claimed in claim 2, characterised in that the amphilphilic, zwitterionic, strongly polar or lipophilic pharmaceutical active ingredient is bamipine maleate, clotrimazole, terbinafine, and acetylsalicylic acid or hydrocortisone acetate.

4. The pharmaceutical formulation as claimed in any one of the foregoing claims, characterised in that it has no other contents.

5. The pharmaceutical formulation as claimed in any one of the foregoing claims, characterised in that the polyetherpolyol has polyoxyethylene units and/or polyoxypropylene units.

6. The pharmaceutical formulation as claimed in any one of the foregoing claims, characterised in that the polyetherpolyol selected is from the group including polyethylene glycols, polypropylene glycols and poloxamers and mixtures thereof, in each case with a molecular weight in the region of 1000 to 30000.

7. The pharmaceutical formulation as claimed in any one of the foregoing claims, characterised in that the lipid is selected from the group containing medium-chain triglyceride, fatty oils, paraffin oil, liquid waxes, isopropyl myristate, cetyl oleate and mixtures thereof.

8. The pharmaceutical formulation as claimed in any one of the foregoing claims, characterised in that the C₂-C₄-alcohol is ethanol, propanol, butanol, tert. butanol and/or isopropanol.

9. The pharmaceutical formulation as claimed in any one of the foregoing claims, characterised in that the polyetherpolyol is poloxamer 407.

10. A process for manufacturing a pharmaceutical formulation as claimed in any one of the foregoing claims.
